(19) United States
(12) Patent Application Publication
(10) Pub. No.: US 2011/0033387 A1
(43) Pub. Date: Feb. 10, 2011

(54) METHOD OF DETERMINATION OF PDH ACTIVITY AND IMAGING MEDIA FOR USE IN SAID METHOD

(75) Inventors: Marie A. Schroeder, Oxfordshire (GB); Lowri E. Cochlin, Oxfordshire (GB); Helen J. Atherton, Oxfordshire (GB); Lisa C. Heather, Oxfordshire (GB); Kieran Clarke, Oxfordshire (GB); George K. Radda, Oxfordshire (GB); Damian J. Tyler, Oxfordshire (GB)

Correspondence Address: GE HEALTHCARE, INC., IP DEPARTMENT 101 CARNEGIE CENTER, PRINCETON, NJ, 08540-6231, US

(73) Assignee: GE HEALTHCARE LIMITED, Little Chalfont Buckinghamshire (GB)

(21) Appl. No.: 12/675,136

(22) PCT Filed: Sep. 5, 2008
(86) PCT No.: PCT/EP2008/061725

§ 371 Date: Oct. 21, 2010

(30) Foreign Application Priority Data
Sep. 7, 2007 (NO) 20074529
Jun. 6, 2008 (EP) 08010318.7

(51) Int. Cl. A61K 49/06; C12Q 1/32

(52) U.S. Cl. 424/9.2; 435/26; 424/9.3

(57) ABSTRACT

The invention relates to a method of determination of PDH activity by ¹³C-MR detection using an imaging medium which comprises hyperpolarised ¹³C-pyruvate and to imaging media for use in said method.

The invention relates to a method of determination of PDH activity by ¹³C-MR detection using an imaging medium which comprises hyperpolarised ¹³C-pyruvate and to imaging media for use in said method.

Within tissues adenosine triphosphate (ATP) provides the energy for synthesis of complex molecules and, in muscle, for contraction. ATP is generated from the metabolism of energy-rich substrates such as glucose or long chain fatty acids. In oxidative tissues such as muscle the majority of the ATP is generated from acetyl-CoA which enters the citric acid cycle, thus the supply of acetyl-CoA is a critical determinant of ATP production in oxidative tissues.

Acetyl-CoA is produced either by β-oxidation of fatty acids or as a result of glucose metabolism by the glycolytic pathway. The key regulatory enzyme in controlling the rate of acetyl-CoA formation from glucose is pyruvate dehydrogenase (PDH) which catalyses the oxidation of pyruvate to acetyl-CoA and carbon dioxide with concomitant reduction of nicotinamide adenine dinucleotide (NAD) to its reduced form (NADH). Thus, PDH is a key enzyme in controlling the rate of oxidative glycolysis and regulating the balance between oxidation of carbohydrate and lipid fuels.

Recently there has been renewed interest in the structure and functioning of the PDH complex, due to realisation that altered PDH complex activity is a feature in many human disorders ranging from the relatively uncommon primary PDH deficiency to major causes of morbidity and mortality, such as diabetes, starvation, sepsis and Alzheimer's disease.

PDH is an intramitochondrial multienzyme complex consisting of multiple copies of several subunits including three enzyme activities E1, E2 and E3, required for the completion of the conversion of pyruvate to acetyl-CoA (Patel et al., FASEB J. 4, 1990, 3224-3233). E1 catalyses the irreversible loss of carbon dioxide from pyruvate; E2 forms acetyl-CoA and E3 reduces NAD to NADH. Two additional enzyme activities are associated with the complex: a specific kinase which is capable of phosphorylating E1 at three serine residues and a loosely-associated specific phosphatase which reverses the phosphorylation. Phosphorylation of a single one of the three serine residues renders the E1inactive. The proportion of the PDH in its active (dephosphorylated) state is determined by a balance between the activity of the kinase (PDH kinase, PDHK) and the phosphatase. The activity of the kinase may be regulated in vivo by the relative concentrations of metabolic substrates such as [NADH]/[NAD⁺], [acetyl-CoA]/[CoA] and [ATP]/[adenosine diphosphate (ADP)] as well as by the availability of pyruvate itself.

The reactions of PDH serve to interconnect the metabolic pathways of glycolysis, gluconeogenesis and fatty acid synthesis to the citric acid cycle. As a consequence, PDH activity is highly regulated by a variety of allosteric effectors and by covalent modification.

In disease states such as Type 1 and Type 2 diabetes, oxidation of lipids is increased with a concomitant reduction in utilisation of glucose, which contributes to hyperglycaemia. Reduced glucose utilisation in both Type 1 and Type 2 diabetes is associated with a reduction in PDH activity. In addition, a further consequence of reduced PDH activity may be that an increase in pyruvate concentration results in increased availability of lactate as a substrate for hepatic gluconeogenesis. It is reasonable to expect that increasing the activity of PDH could increase the rate of glucose oxidation and hence overall glucose utilisation, in addition to reducing hepatic glucose output.

Another factor contributing to diabetes mellitus is impaired insulin secretion, which has been shown to be associated with reduced PDH activity in pancreatic β-cells (Zhou et al., Diabetes 45, 1996, 580-586).

Oxidation of glucose is capable of yielding more ATP per mole of oxygen than is oxidation of fatty acids. In conditions where energy demand may exceed energy supply, such as cardiac failure and certain cardiac myopathies, myocardial ischemia, peripheral vascular disease (including intermittent claudication), cerebral ischemia and reperfusion, muscle weakness, hyperlipidemia, Alzheimer's disease and atherosclerosis, shifting the balance of substrate utilisation in favour of glucose metabolism by elevating PDH activity may be expected to improve the ability to maintain ATP levels and hence function.

As mentioned earlier, the diabetic state should benefit from PDH activation by inhibiting gluconeogenesis and promoting glucose disposal in peripheral tissues. Preliminary evidence in support of this proposal was obtained using dichloroacetate (DCA). The search for novel, small-molecule inhibitors of PDHK offering improved potency and specificity has now been ongoing for several years.

From the aforesaid, it is apparent that the determination of PDH activity plays a key role in the diagnosis of certain disorders and diseases. Further, determining the PDH activity is crucial in assessing treatment response, e.g. response to treatment with drugs which influence, i.e. elevate PDH activity and in drug screening of drugs which impact PDH-activity.

Various methods for the determination of PDH activity are known, which can be grossly divided into in vitro and in vivo tests.

WO-A-2004/021000 discloses antibodies specific for PDH that can be used to immunoprecipitate PDH from a patient sample in an active state. The amount and/or active state of PDH can be determined in vitro in an immunoassay.

In vitro PDH activity tests are further disclosed in WO-A-99/62506. These assays are either in vitro assays with isolated enzymes which include time-consuming preparations like PCR isolation and cloning of PDH kinase or cell assays which require isolation of primary cells.

In vivo PDH activity may be determined in an ex vivo assay by removal of tissue samples (e.g. muscle tissue or liver tissue) which is extracted as described in WO-A-99/62506. A portion of the extract is treated with PDH phosphatase prepared from pig-hearts and the activity of an untreated sample is compared with the activity of the dephosphorylated sample thus prepared by the method of Stansbie et al., Biochem. J. 154 (1976), 225.

Hence there is a need for new and improved methods to determine PDH activity, especially PDH activity in vivo.

It has now been found that hyperpolarised ¹³C-pyruvate can be used as an agent for determining PDH activity in vivo and in vitro by using ¹³C-MR detection.

As mentioned above, pyruvate is a precursor in the citric acid cycle and PDH catalyses the oxidation of pyruvate to acetyl-CoA and carbon dioxide (CO₂), which is in rapid equilibrium with bicarbonate (HCO₃⁻).

It has been found that the metabolic conversion of hyperpolarised ¹³C-pyruvate into its metabolites hyperpolarised ¹³C-lactate, hyperpolarised ¹³C-bicarbonate (in the case of ¹³C₁-pyruvate, ¹³C_{1,2}-pyruvate, ¹³C_{1,3}-pyruvate or ¹³C_{1,2,3}-pyuruvate only) and hyperpolarised ¹³C-alanine can be used to study metabolic processes in the human and non-human animal body using MR. ¹³C₁-pyruvate has a T₁ relaxation in human full blood at 37° C. of about 42 s, however, the conversion of hyperpolarised ¹³C-pyruvate to hyperpolarised ¹³C-lactate, hyperpolarised ¹³C-bicarbonate and hyperpolarised ¹³C-alanine has been found to be fast enough to allow signal detection from the ¹³C-pyruvate parent compound and its metabolites. The amount of alanine, bicarbonate and lactate is dependent on the metabolic status of the tissue under investigation. The MR signal intensity of hyperpolarised ¹³C-lactate, hyperpolarised ¹³C-bicarbonate and hyperpolarised ¹³C-alanine is related to the amount of these compounds and the degree of polarisation left at the time of detection, hence by monitoring the conversion of hyperpolarised ¹³C-pyruvate to hyperpolarised ¹³ C-lactate, hyperpolarised ¹³C-bicarbonate and hyperpolarised ¹³C-alanine it is possible to study metabolic processes in viva in the human or non-human animal body by using non-invasive MR imaging or MR spectroscopy.

It has further been found that the MR signal amplitudes arising from the different pyruvate metabolites varies depending on the tissue type. The unique metabolic peak pattern formed by alanine, lactate, bicarbonate and pyruvate can be used as a fingerprint for the metabolic state of the tissue under examination and thus allows for the discrimination between healthy tissue and tumour tissue. The use of hyperpolarised ¹³C-pyruvate for tumour imaging—with tumour tissue showing high metabolic activity—has been described in detail in WO-A-2006/011810.

Further, the use of hyperpolarised ¹³C-pyruvate for cardiac imaging has been described in WO-A-2006/054903.

Thus, in a first aspect the invention provides a method of determining PDH activity by ¹³C-MR detection using an imaging medium comprising hyperpolarised ¹³C-pyruvate wherein the signal of ¹³C-bicarbonate and optionally ¹³C-pyruvate is detected.

The term “determining PDH activity” denotes the initial measurement of PDH activity including the measurement of the initial rate and the determination of the rate constant.

The term “¹³C-MR detection” denotes ¹³C-MR imaging or ¹³C-MR spectroscopy or combined ¹³C-MR imaging and ¹³C-MR spectroscopy, i.e. ¹³C-MR spectroscopic imaging. The term further denotes ¹³C-MR spectroscopic imaging at various time points.

The term “imaging medium” denotes a liquid composition comprising hyperpolarised ¹³C-pyruvate as the MR active agent, i.e. imaging agent.

The imaging medium used in the method of the invention may be used as an imaging medium for in vivo ¹³C-MR detection, i.e. in living human or non-human animal beings. Further, the imaging medium used in the method of the invention may be used as an imaging medium for in vitro ¹³C-MR detection, e.g. in cell cultures, body samples such as blood or cerebrospinal fluid, ex vivo tissue, for instance ex vivo tissue obtained from a biopsy or isolated organs, all of those derived from a human or non-human animal body.

The term “¹³C-pyruvate” denotes a salt of ¹³C-pyruvic acid that is isotopically enriched with ¹³C, i.e. in which the amount of ¹³C isotope is greater than its natural abundance.

The isotopic enrichment of the hyperpolarised ¹³C-pyruvate used in the method of the invention is preferably at least 75%, more preferably at least 80% and especially preferably at least 90%, an isotopic enrichment of over 90% being most preferred. Ideally, the enrichment is 100%. ¹³C-pyruvate used in the method of the invention has to be isotopically enriched at least at the C1-position (in the following denoted ¹³C₁-pyruvate), since it is the C1-atom of pyruvate which is part of the carbon dioxide (and thus bicarbonate) generated by the PDH-catalysed oxidation of pyruvate. Further, ¹³C-pyruvate used in the method of the invention may be isotopically enriched at the C1- and the C2-position (in the following denoted ¹³C_{1,2}-pyruvate), at the C1- and the C3-position (in the following denoted ¹³C_{1,3}-pyruvate) or at the C1-, C2- and C3-position (in the following denoted ¹³C_{1,2,3}-pyruvate). Isotopic enrichment at the C1-position only is preferred since ¹³C₁-pyruvate is readily available and has a favourably high T₁ relaxation in human full blood at 37° C. (about 42 s).

The terms “hyperpolarised” and “polarised” are used interchangeably hereinafter and denote a nuclear polarisation level in excess of 0.1%, more preferred in excess of 1% and most preferred in excess of 10%.

The level of polarisation may for instance be determined by solid state ¹³C-NMR measurements in solid hyperpolarised ¹³C-pyruvate, e.g. solid hyperpolarised ¹³C-pyruvate obtained by dynamic nuclear polarisation (DNP) of ¹³C-pyruvate. The solid state ¹³C-NMR measurement preferably consists of a simple pulse-acquire NMR sequence using a low flip angle. The signal intensity of the hyperpolarised ¹³C-pyruvate in the NMR spectrum is compared with signal intensity of ¹³C-pyruvate in a NMR spectrum acquired before the polarisation process. The level of polarisation is then calculated from the ratio of the signal intensities of before and after polarisation.

In a similar way, the level of polarisation for dissolved hyperpolarised ¹³C-pyruvate may be determined by liquid state NMR measurements. Again the signal intensity of the dissolved hyperpolarised ¹³C-pyruvate is compared with the signal intensity of a reference sample of known composition, e.g. liquid pyruvic acid or sodium pyruvate dissolved in an aqueous solution. The level of polarisation is then calculated from the ratio of the signal integrals of hyperpolarised ¹³C-pyruvate and the known reference sample, optionally corrected for the relative concentrations. The polarisation can also be determined by comparing with the thermal equilibrium signal of the same ¹³C-pyruvate sample after the hyperpolarisation has died away.

Hyperpolarisation of NMR active ¹³C-nuclei may be achieved by different methods which are for instance described in WO-A-98/30918, WO-A-99/24080 and WO-A-99/35508, which are incorporated herein by reference and hyperpolarisation methods are polarisation transfer from a noble gas, “brute force”, spin refrigeration, the parahydrogen method and dynamic nuclear polarisation (DNP).

To obtain hyperpolarised ¹³C-pyurvate, it is preferred to either polarise ¹³C-pyruvate directly or to polarise ¹³C-pyruvic acid and convert the polarised ¹³C-pyruvic acid to polarised ¹³C-pyruvate, e.g. by neutralisation with a base

One suitable way for obtaining hyperpolarised ¹³C-pyruvate is the polarisation transfer from a hyperpolarised noble gas which is described in WO-A-98/30918. Noble gases having non-zero nuclear spin can be hyperpolarised by the use of circularly polarised light. A hyperpolarised noble gas, preferably He or Xe, or a mixture of such gases, may be used to effect hyperpolarisation of ¹³C-nuclei. The hyperpolarised gas may be in the gas phase, it may be dissolved in a liquid/solvent, or the hyperpolarised gas itself may serve as a solvent. Alternatively, the gas may be condensed onto a cooled solid surface and used in this form, or allowed to sublime. Intimate mixing of the hyperpolarised gas with ¹³C-pyruvate or ¹³C-pyruvic acid is preferred. Hence, if ¹³C-pyruvic acid is polarised, which is a liquid at room temperature, the hyperpolarised gas is preferably dissolved in a liquid/solvent or serves as a solvent. If ¹³C pyruvate is polarised, the hyperpolarised gas is preferably dissolved in a liquid/solvent, which also dissolves pyruvate.

Another suitable way for obtaining hyperpolarised ¹³C-pyruvate is that polarisation is imparted to ¹³C-nuclei by thermodynamic equilibration at a very low temperature and high field. Hyperpolarisation compared to the operating field and temperature of the NMR spectrometer is effected by use of a very high field and very low temperature (brute force). The magnetic field strength used should be as high as possible, suitably higher than 1 T, preferably higher than 5 T, more preferably 15 T or more and especially preferably 20 T or more. The temperature should be very low, e.g. 4.2 K or less, preferably 1.5 K or less, more preferably 1.0 K or less, especially preferably 100 mK or less.

Another suitable way for obtaining hyperpolarised ¹³C-pyruvate is the spin refrigeration method. This method covers spin polarisation of a solid compound or system by spin refrigeration polarisation. The system is doped with or intimately mixed with suitable crystalline paramagnetic materials such as Ni²⁺, lanthanide or actinide ions with a symmetry axis of order three or more. The instrumentation is simpler than required for DNP with no need for a uniform magnetic field since no resonance excitation field is applied. The process is carried out by physically rotating the sample around an axis perpendicular to the direction of the magnetic field. The pre-requisite for this method is that the paramagnetic species has a highly anisotropic g-factor. As a result of the sample rotation, the electron paramagnetic resonance will be brought in contact with the nuclear spins, leading to a decrease in the nuclear spin temperature. Sample rotation is carried out until the nuclear spin polarisation has reached a new equilibrium.

In a preferred embodiment, DNP (dynamic nuclear polarisation) is used to obtain hyperpolarised ¹³C-pyruvate. In DNP, polarisation of MR active nuclei in a compound to be polarized is affected by a polarisation agent or so-called DNP agent, a compound comprising unpaired electrons. During the DNP process, energy, normally in the form of microwave radiation, is provided, which will initially excite the DNP agent. Upon decay to the ground state, there is a transfer of polarisation from the unpaired electron of the DNP agent to the NMR active nuclei of the compound to be polarised, e.g. to the ¹³C nuclei in ¹³C-pyruvate. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. The DNP technique is for example further described in WO-A-98/58272 and in WO-A-01/96895, both of which are included by reference herein.

To polarise a compound by the DNP method, a mixture of the compound to be polarised and a DNP agent is prepared (“a sample”) which is either frozen and inserted as a solid into a DNP polariser for polarisation or which is inserted into a DNP polariser as a liquid and freezes inside said polariser due to the very low surrounding temperature. After the polarisation, the frozen solid hyperpolarised sample is rapidly transferred into the liquid state either by melting it or by dissolving it in a suitable dissolution medium. Dissolution is preferred and the dissolution process of a frozen hyperpolarised sample and suitable devices therefore are described in detail in WO-A-02/37132. The melting process and suitable devices for the melting are for instance described in WO-A-02/36005.

In order to obtain a high polarisation level in the compound to be polarised said compound and the DNP agent need to be in intimate contact during the DNP process. This is not the case if the sample crystallizes upon being frozen or cooled. To avoid crystallization, either glass formers need to be present in the sample or compounds need to be chosen for polarisation which do not crystallize upon being frozen but rather form a glass.

As mentioned earlier ¹³C-pyruvic acid or ¹³C-pyruvate are suitable starting materials to obtain hyperpolarized ¹³C-pyruvate.

Isotopically enriched ¹³C-pyruvate is commercially available, e.g. as sodium ¹³C-pyruvate. Alternatively, it may be synthesized as described by S. Anker, J. Biol. Chem 176, 1948, 133-1335.

Several methods for the synthesis of ¹³C₁-pyruvic acid are known in the art. Briefly, Seebach et al., Journal of Organic Chemistry 40(2), 1975, 231-237 describe a synthetic route that relies on the protection and activation of a carbonyl-containing starting material as an S,S-acetal, e.g. 1,3-dithian or 2-methyl-1,3-dithian. The dithiane is metallated and reacted with a methyl-containing compound and/or ¹³CO₂. By using the appropriate isotopically enriched ¹³C-component as outlined in this reference, it is possible to obtain ¹³C₁-pyruvate or ¹³C_{1,2}-pyruvate. The carbonyl function is subsequently liberated by use of conventional methods described in the literature. A different synthetic route starts from acetic acid, which is first converted into acetyl bromide and then reacted with Cu¹³CN. The nitrite obtained is converted into pyruvic acid via the amide (see for instance S. H. Anker et al., J. Biol. Chem. 176 (1948), 1333 or J. E. Thirkettle, Chem Commun. (1997), 1025). Further, ¹³C-pyruvic acid may be obtained by protonating commercially available sodium ¹³C-pyruvate, e.g. by the method described in U.S. Pat. No. 6,232,497 or by the method described in WO-A-2006/038811.

The hyperpolarisation of ¹³C-pyruvic acid by DNP is described in detail in WO-A1-2006/011809, which is incorporated herein by reference. Briefly, ¹³C-pyruvic acid may be directly used for DNP since it forms a glass when frozen. After DNP, the frozen hyperpolarised ¹³C-pyruvic acid needs to be dissolved and neutralised, i.e. converted to ¹³C-pyruvate. For the conversion, a strong base is needed. Further, since ¹³C-pyruvic acid is a strong acid, a DNP agent needs to be chosen which is stable in this strong acid. A preferred base is sodium hydroxide and conversion of hyperpolarised ¹³C-pyruvic acid with sodium hydroxide results in hyperpolarised sodium ¹³C-pyruvate, which is the preferred ¹³C-pyruvate for an imaging medium which is used for in vivo MR imaging and/or spectroscopy, i.e. MR imaging and/or spectroscopy carried out on living human or non-human animal beings.

Alternatively, ¹³C-pyruvate, i.e. a salt of ¹³C-pyruvic acid can be used for DNP. Preferred salts are those ¹³C-pyruvates which comprise an inorganic cation from the group consisting of NR₄⁺, K⁺, Rb⁺, Cs⁺, Ca²⁺, Sr²⁺ and Ba²⁺, preferably NH₄⁺, K⁺, Rb⁺ or Cs⁺, more preferably K⁺, Rb⁺, Cs⁺ and most preferably Cs⁺, as in detail described in WO-A2-2007/111515 and incorporated by reference herein. The synthesis of these preferred ¹³C-pyruvates is disclosed in WO-A2-20071/111515 as well. If the hyperpolarized ¹³C-pyruvate is used in an imaging medium for in vivo MR imaging and/or spectroscopy it is preferred to exchange the inorganic cation from the group consisting of NH₄⁺, K⁺, Rb⁺, Cs⁺, Ca²⁺, Sr²⁺ and Ba²⁺ by a physiologically very well tolerable cation like Na⁺ or meglumine. This may be done by methods known in the art like the use of a cation exchange column.

Further preferred salts are ¹³C-pyruvates of an organic amine or amino compound, preferably TRIS-¹³C₁-pyruvate or meglumine-¹³C₁-pyruvate, as in detail described in WO-A2-2007/069909 and incorporated by reference herein. The synthesis of these preferred ¹³C-pyruvates is disclosed in WO-A2-2007/069909 as well.

If the hyperpolarised ¹³C-pyruvate used in the method of the invention is obtained by DNP, the sample to be polarised comprising ¹³C-pyruvic acid or ¹³C-pyruvate and a DNP agent may further comprise a paramagnetic metal ion. The presence of paramagnetic metal ions in composition to be polarised by DNP has found to result in increased polarisation levels in the ¹³C-pyruvic acid/¹³C-pyruvate as described in detail in WO-A2-2007/064226 which is incorporated herein by reference.

In another embodiment, the imaging medium used in the method of the invention comprises hyperpolarised ¹³C-pyruvate and malate. Thus, in a second aspect the invention provides a method of determining PDH activity by ¹³C-MR detection using an imaging medium comprising malate and hyperpolarised ¹³C-pyruvate wherein the signal of ¹³C-bicarbonate and optionally ¹³C-pyruvate is detected.

In the context of the invention, the term “malate” denotes a salt of malic acid. The malate is non-hyperpolarised.

Malate is suitably added to the hyperpolarised ¹³C-pyruvate after the polarisation process. Several ways of adding the malate are possible. Where the polarisation process results in a liquid composition comprising the hyperpolarised ¹³C-pyruvate, malate may be dissolved in said liquid composition or a solution of malate in a suitable solvent, preferably an aqueous carrier may be added to the liquid composition. If the polarisation process results in a solid composition comprising the hyperpolarised ¹³C-pyruvate or ¹³C-pyruvic acid, e.g. when DNP has been used, malate may be added to and dissolved in the dissolution medium which is used to dissolve the solid composition. For instance ¹³C-pyruvate polarised by the DNP method may be dissolved in an aqueous carrier like water or a buffer solution containing malate or ¹³C-pyruvic acid polarised by the DNP method may be dissolved in a dissolution medium containing a base to covert pyruvic acid into pyruvate and malate. Alternatively, malate may be added to the final liquid composition, i.e. to the liquid composition after dissolution/melting or to the liquid composition after removal of the DNP agent and/or an optional paramagnetic metal ion. Again the malate may be added as a solid to the liquid composition or preferably dissolved in a suitable solvent, e.g. an aqueous carrier like water or a buffer solution. To promote dissolution of the malate, several means known in the art, such as agitation, stirring, vortexing or sonication may be used. However, methods are preferred which are quick and do not require a mixing device or help coming into contact with the liquid composition.

Suitably, malate is added in the form of malic acid or a salt of malic acid, preferably sodium malate. The concentration of hyperpolarised ¹³C-pyruvate and malate in the imaging medium used in the method of the invention is about equal or malate is present at a lower or higher concentration than ¹³C-pyruvate. If for instance the imaging medium contains x M ¹³C-pyruvate, it contains x M or about x M or less malate but preferably not less than a tenth of x M malate or more malate but preferably not more than three times x M malate. In a preferred embodiment, the concentration of malate in the imaging medium used in the method of the invention is about equal or equal to the concentration of hyperpolarised ¹³C-pyruvate. The term “about equal concentration” denotes a malate concentration which is +/−30% of the concentration of ¹³C-pyruvate, preferably +/−20%, more preferably +/−10%.

By using an imaging medium comprising malate and hyperpolarised ¹³C-pyruvate the nature of PDH regulation can be ascertained. PDH flux can be inhibited by either inactivation of the enzyme complex by PDK, as previously discussed, or also instantaneously by end-product inhibition. Increased NADH/NAD⁺ or acetyl CoA/CoA ratios have been demonstrated to decrease PDH-mediated pyruvate oxidation and oxaloacetate availability for incorporation of acetyl CoA into Krebs cycle is a fundamental determinant of intramitochondrial acetyl CoA concentration. Malate is an intermediate of the oxidative metabolism of glucose, and can enter the Krebs cycle as oxaloacetate via an anaplerotic pathway to increase the overall carbon flux. Without wanting to be bound to this hypothesis, we assume that by administering an imaging medium comprising malate and hyperpolarised ¹³C-pyruvate, the degree of end-product inhibition on PDH could be limited, and in cases of high PDH activity, increase pyruvate flux through the enzyme complex, which can be determined by the method of the invention. In situations of low PDH activity, we would hypothesise that end-product inhibition would be less important and that malate present in the imaging medium would not affect pyruvate flux through the enzyme complex, which can be determined by the method of the invention.

In yet another embodiment, malate is not present in the imaging medium itself but is administered to the subject under investigation, i.e. the living human or non-human animal being, cell culture, body sample such as a blood samples, ex vivo tissue such as tissue obtained form a biopsy or isolated organ prior to administration of the imaging medium used in the method of the invention.

As mentioned earlier, the imaging medium according to the method of the invention may be used as imaging medium for in vivo PDH activity determination by ¹³C-MR detection, i.e. in living human or non-human animal beings. For this purpose, the imaging medium is provided as a composition that is suitable for being administered to a living human or non-human animal body. Such an imaging medium preferably comprises in addition to the MR active agent ¹³C-pyruvate an aqueous carrier, preferably a physiologically tolerable and pharmaceutically accepted aqueous carrier like water, a buffer solution or saline. Such an imaging medium may further comprise conventional pharmaceutical or veterinary carriers or excipients, e.g. formulation aids such as are conventional for diagnostic compositions in human or veterinary medicine.

Further, the imaging medium according to the method of the invention may be used as imaging medium for in vitro PDH activity determination by ¹³C-MR detection, i.e. in cell cultures, body samples such as blood samples, ex vivo tissues such as biopsy tissue or isolated organs. For this purpose, the imaging medium is provided as a composition that is suitable for being added to, for instance, cell cultures, blood samples, ex vivo tissues like biopsy tissue or isolated organs. Such an imaging medium preferably comprises in addition to the MR active agent ¹³C-pyruvate a solvent which is compatible with and used for in vitro cell or tissue assays, for instance DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such an imaging medium but are not required for such a purpose.

If the imaging medium used in the method of the invention is used for in vivo determination of PDH activity, i.e. in a living human or non-human animal body, said imaging medium is preferably administered to said body parenterally, preferably intravenously. Generally, the body under examination is positioned in an MR magnet. Dedicated ¹³C-MR RF-coils are positioned to cover the area of interest. Exact dosage and concentration of the imaging medium will depend upon a range of factors such as toxicity and the administration route. Suitably, the imaging medium is administered in a concentration of up to 1 mmol pyruvate per kg bodyweight, preferably 0.01 to 0.5 mmol/kg, more preferably 0.1 to 0.3 mmol/kg. At less than 400 s after the administration, preferably less than 120 s, more preferably less than 60 s after the administration, an MR imaging sequence is applied, preferably one that encodes the volume of interest in a combined frequency and spatial selective way. The exact time of applying an MR sequence is highly dependent on the volume of interest and the species.

If the imaging medium used in the method of the invention is used for in vitro determination of PDH activity, said imaging medium is 1 mM to 100 mM in ¹³C-pyruvate, more preferably 20 mM to 90 mM and most preferably 40 to 80 mM in ¹³C-pyruvate.

PDH activity can be determined according to the method of the invention by detecting the ¹³C-bicarbonate signal and optionally the ¹³C-pyruvate signal. The determination is based on the following reaction which is illustrated for ¹³C₁-pyruvate; * denotes the ¹³C-label:

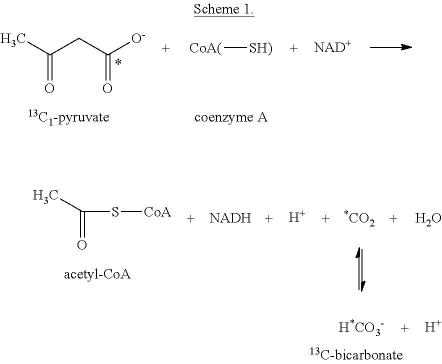

According to scheme 1, a decreased PDH activity manifests itself in a decreased carbon dioxide generation and thus in a decreased ¹³C-bicarbonate signal. At physiological pH the CO₂/bicarbonate equilibrium is shifted towards bicarbonate.

The term “signal” in the context of the invention refers to the MR signal amplitude or integral or peak area to noise of peaks in a ¹³C-MR spectrum which represent ¹³C-bicarbonate and optionally ¹³C-pyruvate. In a preferred embodiment, the signal is the peak area.

In a preferred embodiment, the signals of ¹³C-bicarbonate and ¹³C-pyruvate are detected.

In a preferred embodiment of the method of the invention, the above-mentioned signal of ¹³C-bicarbonate and optionally ¹³C-pyruvate is used to generate a metabolic profile which is an indicator of PDH activity. If the method of the invention is carried out in vivo, i.e. in a living human or non-human animal being, said metabolic profile may be derived from the whole body, e.g. obtained by whole body in vivo ¹³C-MR detection. Alternatively, said metabolic profile is generated from a region or volume of interest, i.e. a certain tissue, organ or part of said human or non-human animal body.

In another preferred embodiment of the method of the invention, the above-mentioned signal of ¹³C-bicarbonate and optionally ¹³C-pyruvate is used to generate a metabolic profile of cells in a cell culture, of body samples such as blood samples, of ex vivo tissue like biopsy tissue or of an isolated organ derived from a human or non-human animal being. Said metabolic profile is then generated by in vitro ¹³C-MR detection.

Thus in a preferred embodiment it is provided a method of determining PDH activity by ¹³C-MR detection using an imaging medium comprising hyperpolarised ¹³C-pyruvate wherein the signal of ¹³C-bicarbonate and optionally ¹³C-pyruvate is detected and wherein said signal or said signals are used to generate a metabolic profile.

In a preferred embodiment, the signals of ¹³C-bicarbonate and ¹³C-pyruvate are used to generate said metabolic profile.

In one embodiment, the spectral signal intensity of ¹³C-bicarbonate and optionally ¹³C-pyruvate is used to generate the metabolic profile. In another embodiment, the spectral signal integral of ¹³C-bicarbonate and optionally ¹³C-pyruvate is used to generate the metabolic profile. In another embodiment, signal intensities from separate images of ¹³C-bicarbonate and optionally ¹³C-pyruvate are used to generate the metabolic profile. In yet another embodiment, the signal intensities of ¹³C-bicarbonate and optionally ¹³C-pyruvate are obtained at two or more time points to calculate the rate of change of ¹³C-bicarbonate and optionally ¹³C-pyruvate.

In another embodiment the metabolic profile includes or is generated using processed signal data of ¹³C-bicarbonate and optionally ¹³C-pyruvate, e.g. ratios of signals, corrected signals, or dynamic or metabolic rate constant information deduced from the signal pattern of multiple MR detections, i.e. spectra or images. Thus, in a preferred embodiment a corrected ¹³C-bicarbonate signal, i.e. ¹³C-bicarbonate to ¹³C-pyruvate signal is included into or used to generate the metabolic profile. In a further preferred embodiment, a corrected ¹³C-bicarbonate to total ¹³C-carbon signal is included into or used to generate the metabolic profile with total ¹³C-carbon signal being the sum of the signals of ¹³C-bicarbonate and ¹³C-pyruvate. In a more preferred embodiment, the ratio of ¹³C-bicarbonate to ¹³C-pyruvate is included into or used to generate the metabolic profile.

The metabolic profile generated in the preferred embodiment of the method according to the invention is indicative for the PDH activity of the body, part of the body, cells, tissue, body sample etc. under examination and said information obtained may be used in a subsequent step for various purposes.

One of these purposes may be the assessment of compounds that alter PDH activity, preferably compounds that elevate PDH activity. A compound that elevates PDH activity may potentially have value in the treatment of disease states associated with disorders of glucose utilisation such as diabetes mellitus, obesity (Curto et al., Int. J. Obes. 21, 1997, 1137-1142) and lactic acidaemia. Additionally such a compound may be expected to have utility in diseases where supply of energy-rich substrates to tissues is limiting such as peripheral vascular disease (including intermittent claudication), cardiac failure and certain cardiac myopathies, muscle weakness, hyperlipidaemias and atherosclerosis (Stacpoole et al., N. Engl. J. Med. 298, 1978, 526-530). A compound that activates PDH may also be useful in treating Alzheimer's disease (Gibson et al., J. Neural. Transm. 105, 1998, 855-870).

In one embodiment, the method of the invention is carried out in vitro and the information obtained is used in assessing the efficacy of potential drugs that alter PDH activity, e.g. in a drug discovery and/or screening process. In such an embodiment, the method of the invention may be carried out in suitable cell cultures or tissue. The cells or the tissue is contacted with the potential drug and PDH activity is determined by ¹³C-MR detection according to the method of the invention. Information about the efficacy of the potential drug may be obtained by comparing the PDH activity of the treated cells or tissue with the PDH activity of non-treated cells or tissue. Alternatively, the variation of PDH activity may be determined by determining the PDH activity of cells or tissue before and after treatment. Such a drug efficacy assessment may be carried out on for instance microplates which would allow parallel testing of various potential drugs and/or various doses of potential drugs and thus would make this suitable for high-throughput screening.

In another embodiment, the method of the invention is carried out in vivo and the information obtained is used in assessing the efficacy of potential drugs that alter PDH activity in vivo. In such an embodiment, the method of the invention may be carried out in for instance test animals or in volunteers in a clinical trial. To the test animal or volunteer a potential drug is administered and PDH activity is determined by ¹³C-MR detection according to the method of the invention. Information about the efficacy of the potential drug may be obtained by determining the variation of PDH activity before and after treatment, e.g. over a certain time period with repeated treatment. Such a drug efficacy assessment may be carried out in pre-clinical research (test animals) or in clinical trials.

In another embodiment, the method of the invention is carried out in vivo or in vitro and the information obtained is used to assess response to treatment and/or to determine treatment efficacy in diseased patients undergoing treatment for their disease. If for instance a patient with diabetes is treated with an anti-diabetic drug that is expected to elevate PDH activity, the PDH activity can be determined according to the method of the invention. Suitably, PDH activity is determined by the method of the invention before commencement of treatment with said anti-diabetic drug and then thereafter, e.g. over a certain time period. By comparing initial PDH activity with the PDH activity during and after the treatment, it is possible to assess whether the anti-diabetic drug shows any positive effect on PDH activity at all and if so, to which extent. To carry out the method of the invention for the above-mentioned purpose in vitro does of course require that suitable samples from a patient under treatment are obtainable, e.g. tissue samples or body samples like blood samples.

As stated earlier the information obtained by the method of the invention may be used in a subsequent step for various purposes.

Another purpose may be to gain insight into disease states, i.e. identifying patients at risk, early detection of diseases, evaluating disease progression, severity and complications related to a disease.

Thus, in one embodiment the method of the invention is carried out in vivo or in vitro and the information obtained is used for identifying patients at risk to develop a disease and/or candidates for preventive measures to avoid the development of a disease. The diagnosis of Type 2 diabetes is often delayed until complications are present (Harris et al., Diabetes Metab. Res. Rev. 16, 2001, 230-236). Early treatment prevents some of the most devastating complications but since current methods of treating Type 2 diabetes remain inadequate, prevention is greatly preferred. Optimal approaches for identifying patients at risk and/or candidates for preventive measures like life-style changes involving low-fat, low-calorie diet and physical activity remain to be determined. Common approaches include glucose tolerance tests and fasting plasma glucose measurements, however patients at risk are not yet hyperglycaemic and hence are not identified by these tests. It would thus be beneficial to have a method which is useful to identify patients at risk to develop Type 2 diabetes and to identify candidates for preventive measures. The method of the invention may provide the necessary information to make that identification. In this embodiment, the method of the invention may be used to determine the initial PDH activity at a first time point and to make subsequent PDH activity determinations over a period of time at a certain frequency, e.g. semi-annually or annually. It can be expected that a decrease in PDH activity will indicate an increasing risk to develop Type 2 diabetes progress and rate of decrease can be used by the physician to decide on commencement of preventive measures and/or treatment. Further, the results of the determination of PDH activity over time could be combined with results from glucose tolerance tests and fasting plasma glucose measurements and the combined results may be used to make a decision on preventive measures and/or treatment. To carry out the method of the invention for the above-mentioned purpose in vitro does of course require that suitable samples from a patient under treatment are obtainable, e.g. tissue samples or body samples like blood samples.

In another embodiment the method of the invention is carried out in vivo or in vitro and the information obtained is used for the early detection of diseases. For several neurodegenerative diseases including Alzheimer's disease, a decreased PDH activity has been reported. For Alzheimer's disease, this effect is specific to certain regions of the brain and it is most prominent in the parietal and temporal lobes. Early diagnosis of such neurodegenerative diseases would allow for early intervention. The method of the invention may provide the necessary information to make that early diagnosis. In this embodiment, the method of the invention may be used to determine the initial PDH activity and compare it with a normal PDH activity, e.g. PDH activity in healthy subjects or to determine the initial PDH activity in certain areas in the brain which are known to be affected by a certain neurodegenerative disease and compare it with PDH activity in areas in the brain which are known to be unaffected by said disease. PDH activity may preferably be used in combination with other clinical markers and/or symptoms characteristic for, e.g. Alzheimer's disease for early diagnosis. To carry out the method of the invention for the above-mentioned purpose in vitro does of course require that suitable samples from a patient under treatment are obtainable, e.g. cerebrospinal fluid.

In yet another embodiment the method of the invention is carried out in vivo or in vitro and the information obtained is used to monitor progression of a disease. This may be useful for diseases or disorders where the disease has not progressed to a level where treatment is indicated or recommended, e.g. because of severe side-effects associated with said treatment. In such a situation the choice of action is “watchful waiting”, i.e. the patient is closely monitored for disease progression and early detection of deterioration. In this embodiment, the method of the invention may be used to determine the initial PDH activity and to make subsequent PDH activity determinations over a period of time at a certain frequency. It can be expected that a decrease in PDH activity will indicate progress and worsening of a disease and the said decrease can be used by the physician to decide on commencement of treatment. To carry out the method of the invention for the above-mentioned purpose in vitro does of course require that suitable samples from a patient under treatment are obtainable, e.g. tissue samples or body samples like blood samples.

In yet another embodiment the method of the invention is carried out in vivo or in vitro and the information obtained is used for determining the severity of a disease. Often diseases progress from their onset over time. Depending on the kind of symptoms and/or the finding of certain clinical markers diseases are characterized by certain stages, e.g. an early (mild) stage, a middle (moderate) stage and a severe (late) stage. More refined stages are common for certain diseases. A variety of clinical markers is known to be used for staging a disease including more specific ones like certain enzymes or protein expression but also more general ones like blood values, electrolyte levels etc. In this context, PDH activity may be such a clinical marker which is used—alone or in combination with other markers and/or symptoms—to determine a disease stage and thus severity of a disease. Hence it may be possible to use the method of the invention for determining PDH activity in a patient in a quantitative way and from the PDH activity value obtained staging the patient's disease. PDH ranges which are characteristic for a certain disease stage may be established by determining PDH activity according to the method of the invention in patients having for instance a disease in an early, middle and late stage and defining a range of PDH activity which is characteristic for a certain stage.

In yet another embodiment the method of the invention is carried out in vivo or in vitro and the information obtained is used for identifying and assessing complications related to a disease. Some diseases, for instance diabetes, can cause many complications, not only acute ones like hypoglycaemia, ketoacidosis or non-ketotic hyperosmolar coma, but also long-term organ-related complications including cardiovascular disease, renal damage and/or failure and retinal damage. Depending on whether and to which degree diabetes affects organs like the heart or the kidney treatment of the disease needs to be modified in such a way to address and reverse these damages. With the method of the invention, PDH activity may be determined in an organ-specific way, for instance by in vivo ¹³C-MR detection carried out with surface coils placed over the heart or the kidney. It can be expected that a low PDH activity in the heart or the kidney is an indicator for said organ being affected by for instance diabetes (Huang et al., Diabetes 52, 2003, 1371-1376).

Since PDH activity is influenced by a variety of factors like dietary status, oxygen availability/status, insulin, and a variety of co-factors, it is important to control these factors, e.g. by providing patients with a diet plan or standardized meals prior to carrying out the method of the invention. Also, it has been found that the patient is not fasted since this would result in a decreased ¹³C-bicarbonate signal.

In one aspect of the invention, the PDH activity is purposely modulated in a controlled way by oral or parenteral administration of for instance glucose, fatty acids or ketone bodies. Oxygen status can be modulated by affecting the breathing gas prior to carrying out the method of the invention or pharmaceutically by inducing stress or changing perfusion.

In another embodiment PDH activity is determined by the method described, but prior, sequential or simultaneous to the quantification of fatty acid metabolism. As described previously acetyl-CoA is generated from glycolysis or fatty acid metabolism, and a shift from one to the other is part of many disease states. In addition to directly determining the PDH activity by method of the invention, the indirect measure of PDH activity by measuring fatty acid metabolism would be complementary and valuable. Fatty acid metabolism may be quantified by administration of an imaging medium comprising hyperpolarised ¹³C-acetate and ¹³C-MR detecting signals from the metabolite ¹³C-acetylcarnitine and optionally ¹³C-acetyl-CoA or ¹³C-acetyl-CoA and the parent compound ¹³C-acetate.

Thus, another aspect of the invention is a method of determining PDH activity by ¹³C-MR detection using an imaging medium comprising hyperpolarised ¹³C-pyruvate and hyperpolarised ¹³C-acetate, wherein the signals of ¹³C-bicarbonate and optionally 13C-pyruvate and the signals of ¹³C-acetylcamitine and optionally ¹³C-acetyl-CoA or ¹³C-acetyl-CoA and ¹³C-acetate are detected.

Yet another aspect of the invention is a method of determining PDH activity by ¹³C-MR detection using an imaging medium comprising hyperpolarised ¹³C-pyruvate wherein the signals of ¹³C-bicarbonate and optionally ¹³C-pyruvate are detected and wherein prior or subsequent to this ¹³ C-MR detection a ¹³C-MR detection is carried out using an imaging medium that comprises hyperpolarised ¹³C-acetate and wherein signals of ¹³C-acetylcamitine and optionally ¹³C-acetyl-CoA or ¹³C-acetyl-CoA and ¹³C-acetate are detected.

¹³C-pyruvate and ¹³C-acetate may be hyperpolarised and administered simultaneously since an imaging medium comprising hyperpolarised ¹³C-pyruvate and hyperpolarised ¹³C-acetate is expected to give a more accurate and complete determination of PDH activity.

Anatomical and/or—where suitable—perfusion information may be included in the method of the invention when carried out in vivo. Anatomical information may for instance be obtained by acquiring a proton or ¹³C-MR image with or without employing a suitable contrast agent before or after the method of the invention.

An MR imaging medium comprising malate and hyperpolarised ¹³C-pyruvate as discussed earlier is novel, thus in yet another aspect the invention provides a MR imaging medium comprising malate and hyperpolarised ¹³C-pyruvate.

Further, an imaging medium comprising hyperpolarised ¹³C-pyruvate and hyperpolarised ¹³C-acetate as discussed earlier is novel as well, thus, in yet another aspect the invention provides a MR imaging medium comprising ¹³C-pyruvate and hyperpolarised ¹³C-acetate.

As mentioned and discussed in detail above, the MR imaging media according to the invention, i.e. the MR imaging medium comprising malate and hyperpolarised ¹³C-pyruvate and MR imaging medium comprising ¹³C-pyruvate and hyperpolarised ¹³C-acetate can be used in a method of determining PDH activity by ¹³C-MR detection.

The imaging media according to the invention may be used as imaging media in vivo, i.e. in living human or non-human animal beings. For this purpose, the imaging media are provided as a composition that is suitable for being administered to a living human or non-human animal body. Such imaging media preferably comprise in addition to the MR active agent ¹³C-pyruvate or ¹³C-pyruvate and ¹³C-acetate or malate and the MR active agent ¹³C-pyruvate an aqueous carrier, preferably a physiologically tolerable and pharmaceutically accepted aqueous carrier like water, a buffer solution or saline. Such imaging media may further comprise conventional pharmaceutical or veterinary carriers or excipients, e.g. formulation aids such as are conventional for diagnostic compositions in human or veterinary medicine.

Further, the imaging media according to the invention may be used as imaging media in vitro, i.e. in cell cultures, body samples such as blood samples, ex vivo tissues such as biopsy tissue or isolated organs. For this purpose, the imaging media are provided as compositions that are suitable for being added to, for instance, cell cultures, blood samples, ex vivo tissues like biopsy tissue or isolated organs. Such imaging media preferably comprise in addition to the MR active agent ¹³C-pyruvate or ¹³C-pyruvate and ¹³C-acetate or malate and the MR active agent ¹³C-pyruvate a solvent which is compatible with and used for in vitro cell or tissue assays, for instance DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such imaging media but are not required for such a purpose.

### BRIEF DESCRIPTION OF THE DRAWINGS:

FIG. 1 shows a comparison of the ratio of the ¹³C-bicarbonate to ¹³C-pyruvate peak amplitude before (“STZ-pre”) and after (“STZ-post”) STZ injection in rats to induce a model of Type 1 diabetes. “*” denotes p=0.01.

FIG. 2 shows the effect of starvation (“fasted”) on the ratio of the ¹³C-bicarbonate to ¹³C-pyruvate peak amplitude in rats. “**” denotes p<0.0001.

FIG. 3 shows the change of the ratio of the ¹³C-bicarbonate to ¹³C-pyruvate peak amplitude over time (2 weeks and 4 weeks) for rats on a high-fat diet (“HFF”) compared to baseline. “#” denotes p<0.002, “##” denotes p<0.005.

FIG. 4 shows the ratio of active/total PDH (%) for the fed rats (“ControlFed”), the starved rats (“ControlFasted”), the rats on a high-fat diet (“High Fat Fed”) and the diabetic rats (“STZ”).

FIG. 5 shows a correlation between the PDH activity measured on the ex vivo heart tissue (protocol previously described by Seymour et at (Seymour, A. M. & Chatham, J. C. (1997) J Mal Cell Cardiol 29, 2771-2778.) and the determination of PDH activity according to the method of the invention by measuring the ratio of the ¹³C-bicarbonate to ¹³C-pyruvate peak amplitude.

FIG. 6 shows in its upper part single average MR spectra acquired in rats before (“Baseline”) and after inducement of hyperthyroidism (7 day—T3) and compared to the MR spectra acquired in a control group (7 day—Control). In the tower part of FIG. 6, the comparison of the ratio of the ¹³C-bicarbonate to ¹³C-pyruvate peak amplitude on day 7 of the diseased group (T3) and the control group (Control) is shown (gray triangles) compared to baseline (black diamonds).

FIG. 7 shows the comparison of ratio of the ¹³C-bicarbonate to ¹³C-pyruvate in fed and fasted rats, following an injection of hyperpolarised ¹³C-pyruvate (light gray bars) or a mixture of hyperpolarised ¹³C-pyruvate and malate (dark gray bars).

### EXAMPLES

In the following the terms pyruvate, ¹³C-pyruvate and ¹³C₁-pyruvate are used interchangeably and all denote ¹³C₁-pyruvate. Likewise the terms pyruvic acid, ¹³C-pyruvic acid and ¹³C₁-pyruvic acid are used interchangeably and all denote ¹³C₁-pyruvic acid.

### Example 1

### Production of an Imaging Medium Comprising Hyperpolarised ¹³C₁-Pyruvate Obtained by the DNP Method

Tris(8-carboxy-2,2,6,6-(tetra(hydroxyethyl)-benzo-[1,2-4,5′]-bis-(1,3)-dithiole-4-yl)-methyl sodium salt (trityl radical) which had been synthesised according to Example 7 of WO-A1-98/39277 was added to ¹³C-pyruvic acid (40 mM) in a test tube to result in a composition being 15mM in trityl radical. Further, an aqueous solution of the Gd-chelate of 1,3,5-tris-(N-(DO3A-acetamido)-N-methyl-4-amino-2-methyl-phenyl)-[1,3,5]tria-zinane-2,4,6-trione (paramagnetic metal ion) which had been synthesised according to Example 4 of WO-A-2007/064226 was prepared and 0.8 (14.6 mM) were added to the test tube with the ¹³C₁-pyruvic acid and the trityl radical.

The composition was transferred from the test tube to a sample cup and the sample cup was inserted into a DNP polarises. The composition was polarised under DNP conditions at 1.2 K in a 3.35 T magnetic field under irradiation with microwave (93.89 GHz) for 45 min.

The composition was subsequently dissolved in an aqueous solution of sodium hydroxide, TRIS buffer and EDTA at a pressure of 10 bar and temperature of 170° C. The resultant imaging medium contained 80 mM of hyperpolarized sodium ¹³C₁-pyruvate at pH 7.2-7.9, with a polarization of about 30% during administration.

### Example 2

### Determination of PDH Activity According to the Method of the Invention in Diabetes Disease Animal Models

Three groups of male Wistar rats were included in this study, to investigate both Type I diabetes and insulin resistance, a precursor to Type II diabetes.

Initial PDH activity (baseline) was determined in a first group of 6 rats according to Example 3. Type I diabetes was subsequently induced in all rats with a single intraperitoneal injection of freshly prepared Streptozotocin (STZ; 50 mg/kg body weight) in 50 mM cold citrate buffer (pH 4.5). Five days after STZ-diabetes induction, PDH activity was determined again, each rat served as its own experimental control. The comparison of the ratio of the ¹³C-bicarbonate to ¹³C-pyruvate peak amplitude before and after STZ injection clearly shows the decrease in said ratio and thus a decrease in PDH activity (FIG. 1)

Rats were then recovered and sacrificed 1 h later by an intraperitoneal injection of sodium pentobarbital for tissue preparation and blood plasma analysis. Heart, lung, liver, and soleus and gastrocnemius muscles were rapidly dissected out, frozen immediately using N₂ cooled aluminium tongs, and stored at −80° C. for later analysis. Approximately 3 ml of blood was drawn from the chest cavity after the heart was excised. Blood was immediately centrifuged (3,200 rpm for 10 min at 4° C.) and plasma was removed. A 200 μl aliquot of plasma was separated and the lipoprotein lipase inhibitor tetrahydrolipostatin (THL) added for nonesterified fatty acid (NEFA) analysis. All plasma samples were immediately frozen and stored at −80° C. An ABX Pentra 400 (Horiba ABX Diagnostics, Montpelier, France) was used to perform assays for plasma glucose, NEFAs (Wako Diagnostics, Richmond, USA) and 3-β-hydroxybutyrate (Randox, Co. Antrim, UK). Plasma insulin was measured using a rat insulin ELISA (Mercodia, Uppsala, Sweden).

The second group of rats (n=12) were split into 2 subgroups and in each subgroup initial PDH activity (baseline) was determined according Example 3.

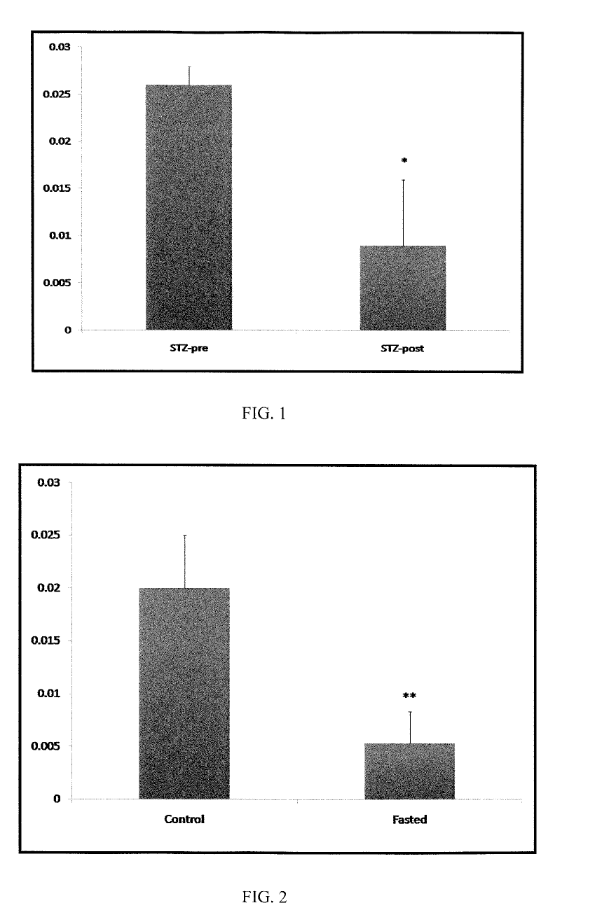

The first subgroup (“fasted”) was fasted overnight prior to each PDH activity determination, with food removed at 1800 hrs on the day before the determination. This corresponded with starvation for 14-18 hrs from the time food was removed. The effect of starvation on the ratio of the ¹³C-bicarbonate to ¹³C-pyruvate peak amplitude is shown in FIG. 2.

In the second subgroup (“fed”), PDH activity was determined in the fed state with food provided ad libitum. After baseline PDH activity determination, all rats were recovered and sacrificed 1 h later for tissue preparation and plasma analysis, as described above.

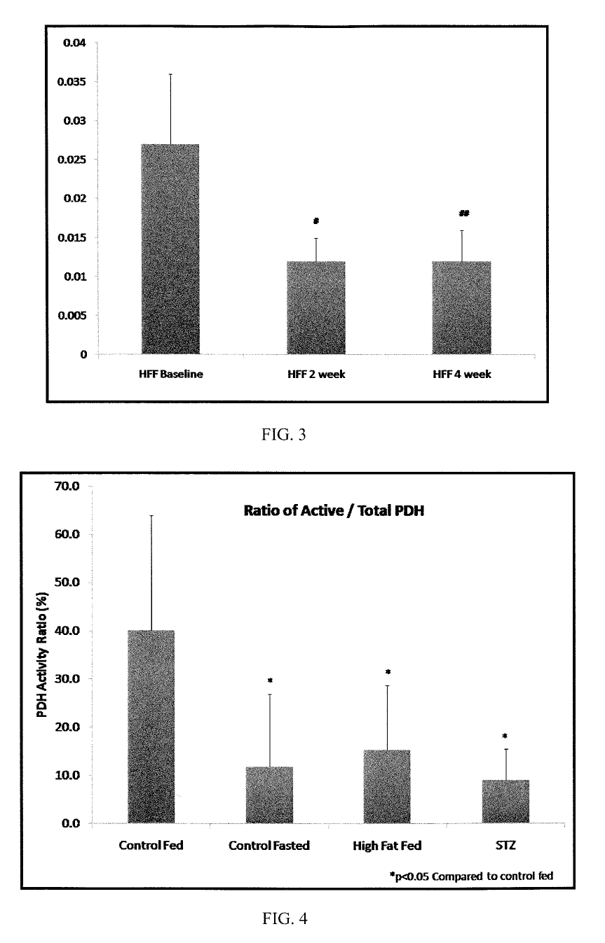

In the third group of rats (n-7) PDH activity was determined according Example 3 at 3 time points: initial PDH activity (baseline), 2 and 4 weeks. After initial PDH activity determination (baseline), all 7 rats were placed on a high fat diet, comprised of 55% of calories from saturated fat, to induce a model of metabolic syndrome, a precursor of Type 2 diabetes. Food was always available ad libitum. After PDH activity determination at the 4 week time point, rats were recovered and sacrificed 1 h later for tissue preparation and plasma metabolite levels, as described above. FIG. 3 shows the change of the ratio of the ¹³C-bicarbonate to ¹³C-pyruvate peak amplitude over time.

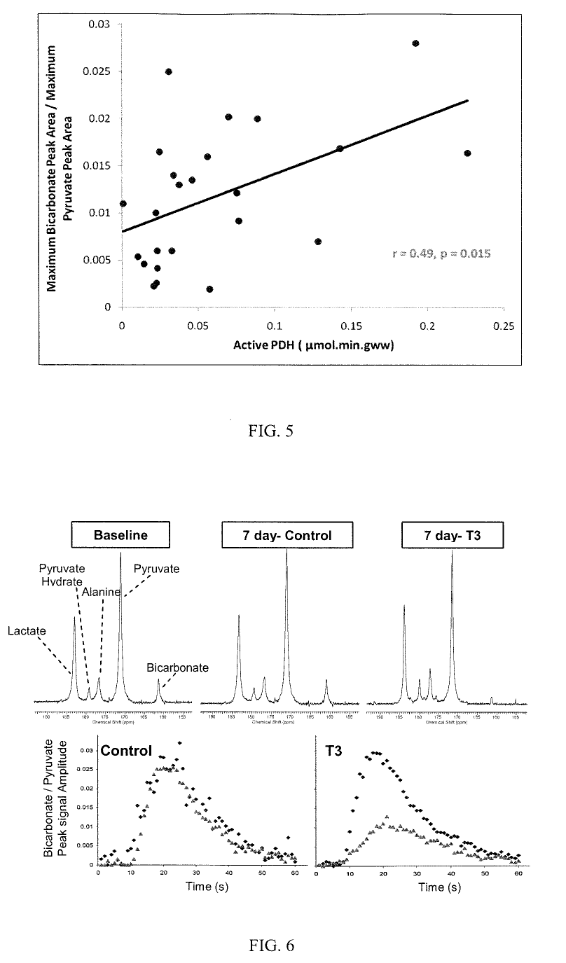

Heart tissue from all animals was analysed to determine the active and total fractions of the PDH enzyme (PDHₐ and PDHₜ) according to the protocol previously described by Seymour et al (Seymour, A. M. & Chatham, J. C. (1997) J Mol Cell Cardiol 29, 2771-2778.) FIG. 4 shows the ratio of the PDH enzyme in the active form. Strong agreement can be seen with the PDH activity results as measured by the ratio of the ¹³C-bicarbonate to ¹³C-pyruvate peak amplitudes in all three groups. This is further emphasized by FIG. 5 which shows a strong correlation between the PDH activity measured on the ex vivo heart tissue and that measured by the ratio of the ¹³C-bicarbonate to ¹³C-pyruvate peak amplitude.

### Example 3

### ¹³ C-MR Detection

### Example 3a

### Animal Preparation

All rats were anaesthetised using isofluorane (2% in oxygen) and kept on a heated mat. Care was taken to maintain body temperature at 37° C. A catheter was introduced into the tail vein, and rats were then placed in a home-built animal handling system. ECG, respiration rate, and body temperature were monitored, and air heating was provided. Anaesthesia was continued by means of isofluorane (1.7%) delivered to a nose cone.

### Example 3b

### Hyperpolarised ¹³C-Pyruvate Dosing and Administration

1 cm³ of the imaging medium as prepared in Example 1 was injected over 10 s via the tail vein catheter in the anaesthetised rat.

### Example 3c

### ¹³C-MR Imaging/Spectroscopy

A home-built ¹H/¹³C butterfly coil was fit over the rat chest, localising signal from the heart. Rats were positioned in a 7 T horizontal bore MR scanner interfaced to a Varian Inova console. Correct positioning was confirmed by the acquisition of an axial proton FLASH image (TE/TR=1.17/2.33 ms, Matrix size=64×64, FOV=60×60 mm, Slice thickness=2.5 mm, Excitation flip angle=15°). A cardiac-gated shim was used to reduce the proton line width to approximately 120 Hz.

Immediately prior to injection, an ECG gated ¹³C-MR pulse-acquire spectroscopy sequence was initiated. 60 individual cardiac spectra were acquired over 1 minute following injection (TR=1 s, Excitation flip angle=5°, Sweep width=6000 Hz, Acquired points=2048, Frequency centred on pyruvate signal).

The series of cardiac ¹³C MR spectra were analysed using the AMARES algorithm as implemented in the jMRUI software package (Naressi et al., Computers in Biology and Medicine, 31(4), 2001, 269-286 and Naressi et al., Magnetic Resonance Materials in Physics, Biology and Medicine, 12(2-3), 2001, 141-152). Spectra were conjugated, and then baseline and DC corrected based on the last half of acquired points. Peaks corresponding with pyruvate and bicarbonate were fitted with prior knowledge assuming a Lorentzian line shape, peak frequencies, relative phases and line widths.

Maximum pyruvate peak area was calculated for each series of spectra, and was used to calculate maximum bicarbonate/pyruvate ratio. This effectively normalized variations in polarisation between each data set. Parameters describing the kinetic progression of bicarbonate, namely time to appearance, time to maximum, and decay time to half maximum were also calculated.

### Example 4

### PDH Activity Determination According to the Method of the Invention in Hyperthyroid Disease Animal Models

Twelve male Wistar rats (2 groups of 6) were included in this study, to investigate the effects of hyperthyroidism on cardiac metabolism.

Initial PDH activity (baseline) was determined in all rats according to Example 3. Hyperthyroidism was subsequently induced in 6 rats with 7 daily intraperitoneal injections of freshly prepared tri-iodothyronine (T3; 0.2 mg/kg body weight/day). The other six rats received 7 daily intraperitoneal injections of saline water (0.9%) to serve as controls. After 7 days of T3 administration, PDH activity was again determined in each of the 12 rats according to the method of the invention. The ¹³C-bicarbonate to ¹³C-pyruvate peak amplitude ratio was compared in rats administered T3 versus control rats both at baseline and day 7. The results clearly show that T3 administration causes a decrease in the ratio of ¹³C-bicarbonate to ¹³C-pyruvate peak amplitude, and this represents a decrease in the activity of PDH (FIG. 6)

Rats were sacrificed 24 h later by an intraperitoneal injection of sodium pentobarbital for tissue preparation. Hearts were rapidly dissected out and cut into two approximately equal halves. One half was frozen immediately using N₂ cooled aluminium tongs, and stored at −80° C. for later biochemical analysis. Intact mitochondria were isolated from the other half of the heart and were used to assess mitochondrial function.

### Example 5

### Determination of PDH Activity According to the Method of the Invention Using an Imaging Medium Comprising Malate and Hyperpolarised 13C-Pyruvate

Six male Wistar rats were examined under each of 4 experimental conditions to determine if infusion of hyperpolarised ¹³C-pyruvate could non-invasively assess the nature of PDH regulation.

In this example, an imaging medium comprising malate and hyperpolarised ¹³C-pyruvate was used to ascertain the nature of PDH regulation. PDH flux can be inhibited by either inactivation of the enzyme complex by PDK or also instantaneously by end-product inhibition. Increased NADH/NAD⁺ or acetyl CoA/CoA ratios have been demonstrated to decrease PDH-mediated pyruvate oxidation, and of course, oxaloacetate availability for incorporation of acetyl CoA into Krebs cycle is a fundamental determinant of intramitochondrial acetyl CoA concentration. Malate is an intermediate of the oxidative metabolism of glucose, and can enter the Krebs cycle via an anaplerotic pathway to increase the overall carbon flux. It was hypothesized that using an imaging medium comprising malate and hyperpolarised ¹³C-pyruvate, the degree of end-product inhibition on PDH could be reduced, In cases of high PDH activity, this would increase pyruvate flux through the enzyme complex, as determined by ¹³C-bicarbonate detection with ¹³C-MR. In fasted rats, due to the already very low PDH activity, it was anticipated that end-product inhibition was irrelevant and that malate co-infusion would not affect the ¹³C-bicarbonate production detected.

Each of 6 rats were examined, according to the protocol described in Example 3, in the fed and fasted states (to modulate PDH activity), with 40 μmol hyperpolarised ¹³C-pyruvate alone and 40 μmol hyperpolarised ¹³C-pyruvate co-infused with 40 μmol malate (to manipulate Krebs cycle flux/acetyl CoA uptake). The imaging medium comprising hyperpolarised ¹³C-pyruvate or malate and hyperpolarised ¹³C-pyruvate was infused via the tail vein into the rats in an MR scanner and cardiac spectra were acquired every second for 1 min. Signals of ¹³C-pyruvate and ¹³C-bicarbonate were detected, conversion of ¹³C-pyruvate to ¹³C-bicarbonate was monitored and the pyruvate to bicarbonate ratio was used as a marker of PDH flux.

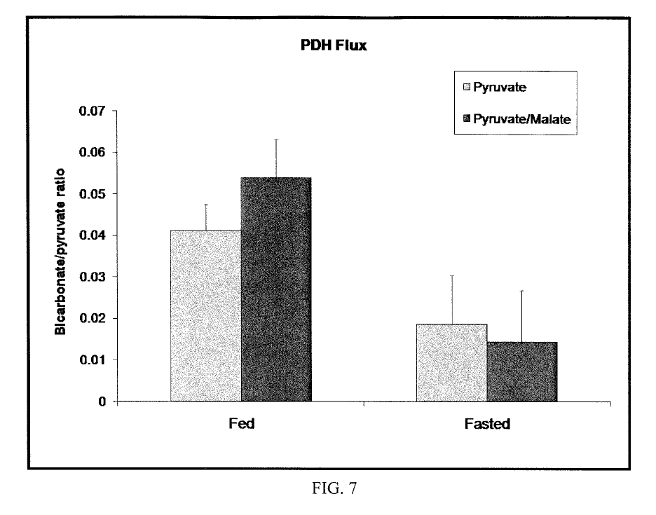

Infusion of the imaging medium comprising malate and hyperpolarised ¹³C-pyruvate increased PDH flux by 32% compared with the imaging medium comprising hyperpolarised ¹³C-pyruvate alone, indicating that removal of acetyl CoA by incorporation into the Krebs cycle increased PDH flux. PDH flux was 55% lower in fasted rats injected with hyperpolarised ¹³C-pyruvate alone compared with fed rats, and did not change when the imaging medium comprising malate and hyperpolarised ¹³C-pyruvate was used. Here, low PDH activity prevented additional enzyme flux. These results, depicted in FIG. 7, suggest that end product inhibition limits fed state PDH flux, whereas PDH activity regulates pyruvate oxidation in the fasted state. In conclusion, this study has provided evidence that hyperpolarised MR may be useful to obtain details of metabolic regulation, rather than just obtaining information about the metabolic state.

1. Method of determining pyruvate dehydrogenase (PDH) activity by ¹³C-MR detection using an imaging medium comprising hyperpolarised ¹³C-pyruvate wherein the signal of ¹³C-bicarbonate and optionally the signal of ¹³C-pyruvate are detected.

2. Method according to claim 1 wherein the signals of ¹³C-bicarbonate and ¹³C-pyruvate are detected.

3. Method according to claim 1 wherein said method is a method of in vivo determination of PDH activity in a human or non-human animal being.

4. Method according to claim 1 wherein said method is a method of in vitro determination of PDH activity in a cell culture, in body samples, in ex vivo tissue or in an isolated organ derived from a human or non-human animal being.

5. Method according to claim 1 wherein said signal or signals are used to generate a metabolic profile.

6. Method according to claim 5 which is carried out in vivo or in vitro and wherein said metabolic profile is used in assessing the efficacy of potential drugs that alter PDH activity.

7. Method according to claim 5 which is carried out in vivo or in vitro and wherein said metabolic profile is used to assess response to treatment and/or to determine treatment efficacy in diseased patients undergoing treatment for their disease.

8. Method according to claim 5 which is carried out in vivo or in vitro and wherein said metabolic profile is used for identifying patients at risk to develop a disease and/or candidates for preventive measures to avoid the development of a disease.

9. Method according to claim 5 which is carried out in vivo or in vitro and said metabolic profile is used for the early detection of diseases.

10. Method according to claim 5 which is carried out in vivo or in vitro and said metabolic profile is used to monitor progression of a disease, determining the severity of a disease or for identifying and assessing complications related to a disease.

11. (canceled)

12. (canceled)

13. Method according to claim 1 wherein said imaging medium further comprises hyperpolarised ¹³C-acetate and wherein in addition signals of ¹³C-acetylcarnitine and optionally ¹³C-acetyl-CoA or ¹³C-acetyl-CoA and ¹³C-acetate are detected.

14. Method according to claim 1 wherein prior or subsequent to said ¹³C-MR detection using an imaging medium comprising hyperpolarised ¹³C-pyruvate, a ¹³C-MR detection is carried out using an imaging medium that comprises hyperpolarised ¹³C-acetate and wherein signals of ¹³C-acetylcarnitine and optionally ¹³C-acetyl-CoA or ¹³C-acetyl-CoA and ¹³C-acetate are detected.

15. Method according to claim 1 wherein said imaging medium further comprises malate.

16. MR imaging medium comprising malate and hyperpolarised ¹³C-pyruvate.

17. MR imaging medium comprising hyperpolarised ¹³C-pyruvate and hyperpolarised ¹³C-acetate.
